(19) United States
(12) Patent Application Publication
(10) Pub. No.: US 2022/0370658 A1
(43) Pub. Date: Nov. 24, 2022

(54) DEVICE TO DISINFECT ITEMS ON A CONVEYOR USING ULTRAVIOLET LIGHT

(71) Applicant: Steribin, LLC, St. George, UT (US)

(72) Inventors: Wayne Provost, Ivins, UT (US); Jonathan M. Cole, St. George, UT (US); Jeffrey Mel Glauser, Santa Clara, UT (US)

(73) Assignee: Steribin, LLC, St. George, UT (US)

(21) Appl. No.: 17/745,543

(22) Filed: May 16, 2022

Related U.S. Application Data
(60) Provisional application No. 63/190,366, filed on May 19, 2021.

(51) Int. Cl. A61L 2/10; B65G 45/10; B65G 23/04; B65G 23/24

(52) U.S. Cl. CPC A61L 2/10; B65G 45/10; B65G 23/04; B65G 23/24; A61L 2202/11; A61L 2202/122; B65G 2812/02148; B65G 2812/02168

(57) ABSTRACT

A disinfecting device using ultraviolet light is configured to sit on top of an existing conveyor belt or rollers. The disinfecting device has a housing, an inlet, an outlet, a plurality of rollers within the housing, one or more disinfecting lights positioned above the rollers, and at least one disinfecting light positioned beneath the rollers. The plurality of rollers carry the item upward, passing over at least one disinfecting light while other disinfecting lights are positioned above the item. The item is then returned to the conveyor belt to continue onward.

### CROSS-REFERENCE TO RELATED APPLICATIONS

This application claims priority to U.S. Provisional Application Ser. No. 63/190,366, filed on May 19, 2021, which is incorporated herein by reference.

### TECHNICAL FIELD

The present disclosure relates to a device to disinfect items. More particularly, the present disclosure relates to a device for disinfecting items on a conveyor.

### BACKGROUND

Infectious diseases commonly spread through the direct transfer of bacteria, viruses, or other microbes from contact with contaminated surfaces. Accordingly, disinfecting surfaces of items is important to maintaining health throughout the world. When surfaces are not disinfected to remove the viruses and bacteria thereon, people may become ill. Manufacturers and others have sought to disinfect items while they pass on conveyor belts. However, current disinfectant systems in the art require modifications to the conveyor system and are customized for the particular conveyor and goods thereon. This makes the installation expensive and burdensome. Additionally, it may require the conveyor to be shut down for installation, costing the company production and loss of money. As a result, companies who have conveyor systems do not want to make modifications to their line due to the high cost and cumbersome process. Also, unless the conveyor system is significantly modified, current disinfection devices are only able to disinfect from the top side of the item (above the conveyor).

Accordingly, there is a need for a disinfectant device for a conveyor belt that may be retrofitted to existing conveyor belts without requiring changes to the conveyor belt or its flow of products, and that is capable of disinfecting all surfaces of an item. The present disclosure seeks to solve these and other issues.

### SUMMARY OF EXAMPLE EMBODIMENTS

In some embodiments, a disinfecting device for conveyors comprises a housing, an inlet, an outlet, a plurality of rollers within the housing, one or more disinfecting lights positioned above the rollers, and at least one disinfecting light positioned beneath the rollers.

In some embodiments, the rollers are driven via the conveyor belt on which the disinfecting device is positioned, the rollers being coupled to one another via a series of belts, chains, etc. In some embodiments, the rollers are driven via at least one motor.

In one method of use, a disinfecting device for conveyors is positioned to rest atop a conveyor belt system. A drive roller, driven by the conveyor belt or motor, drives a plurality of rollers. As an item passes down the conveyor belt, it enters the disinfecting device via the inlet. The plurality of rollers carry the item upward, passing over at least one disinfecting light while other disinfecting lights are positioned above the item. Therefore, the item is exposed to disinfecting light from above and below. It will be appreciated that additional disinfecting lights may be positioned on one or more sides of the housing as well to ensure every surface of the item is disinfected. By placing the disinfecting device atop an existing conveyor belt, no modifications to the conveyor belt are required, minimizing down time while remaining inexpensive to implement. Further, when the rollers are driven by the conveyor belt the impact to production speed is minimal.

In some embodiments, the disinfecting device comprises light-blocking drapes or shields that prevent too much light from being visible to workers who may be working nearby.

### BRIEF DESCRIPTION OF THE DRAWINGS

FIG. 1 illustrates a side, top perspective view of a disinfecting device for conveyors;

FIG. 2 illustrates a side, top perspective view of a disinfecting device for conveyors with a housing made transparent for ease of view of interior components;

FIG. 3 illustrates a detailed, right side elevation view of a disinfecting device for conveyors with the housing removed;

FIG. 4 illustrates a front, detailed perspective view of a disinfecting device for conveyors;

FIG. 5 illustrates a rear elevation view of a disinfecting device for conveyors;

FIG. 6 illustrates a detailed, side elevation view of rollers of a disinfecting device for conveyors;

FIG. 7 illustrates a detailed, side elevation view of rollers of a disinfecting device for conveyors; and

FIG. 8 illustrates a side, top perspective view of a disinfecting device for conveyors with a housing made transparent for ease of view of interior components.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.). While ultraviolet (UV) light is used as an example throughout, it will be appreciated that any light, or electromagnetic wavelength of light, capable of destroying or inhibiting the growth of microorganisms is contemplated herein as a “disinfecting light.”

As previously discussed, there is a need for a disinfectant device for a conveyor belt that may be retrofitted to existing conveyor belts without requiring changes to the conveyor belt or its flow of products, and that is capable of disinfecting all surfaces of an item. The disinfecting device for conveyors disclosed herein solves these and other problems.

In some embodiments, generally, a disinfecting device for conveyors is configured to sit on top of an existing conveyor system (e.g., conveyor belt, rollers, etc.). This allows the disinfecting device for conveyors to be used on nearly any conveyor system without having to modify the conveyor line. As a result, a company wishing to disinfect items passing on its conveyor belt may simply place the disinfecting device for conveyors on the conveyor where convenient and where a power source is available for the internal disinfecting lights (e.g., UV lights). As a result, the downtime of the conveyor system and the overall impact to production speeds is minimal, overcoming problems in the prior art. Additionally, because the disinfecting device for conveyors is capable of disinfecting from below the items, it likewise overcomes limitations in the prior art. While conveyor belts are used as an example throughout, it will be appreciated that any type of conveyor may be used, such as a roller conveyor.

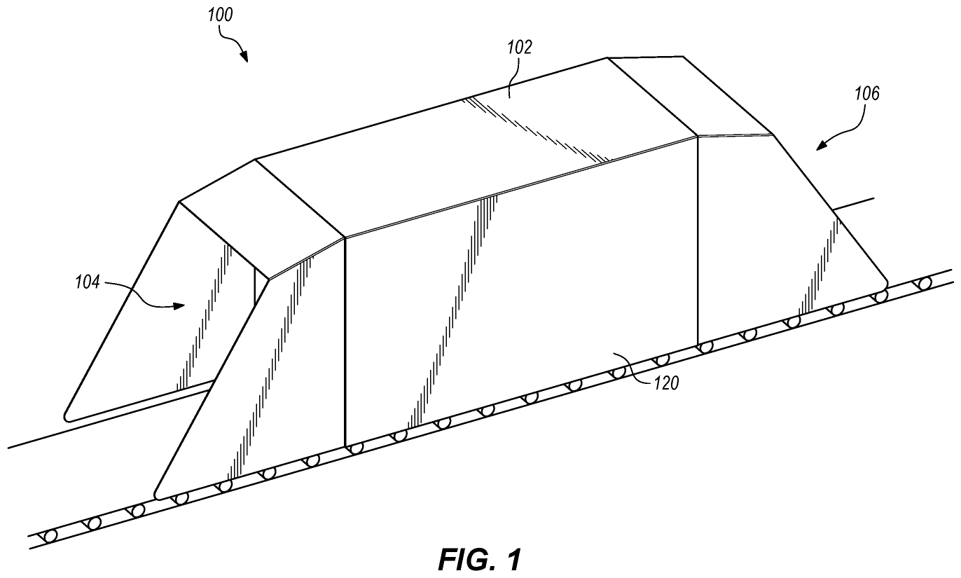

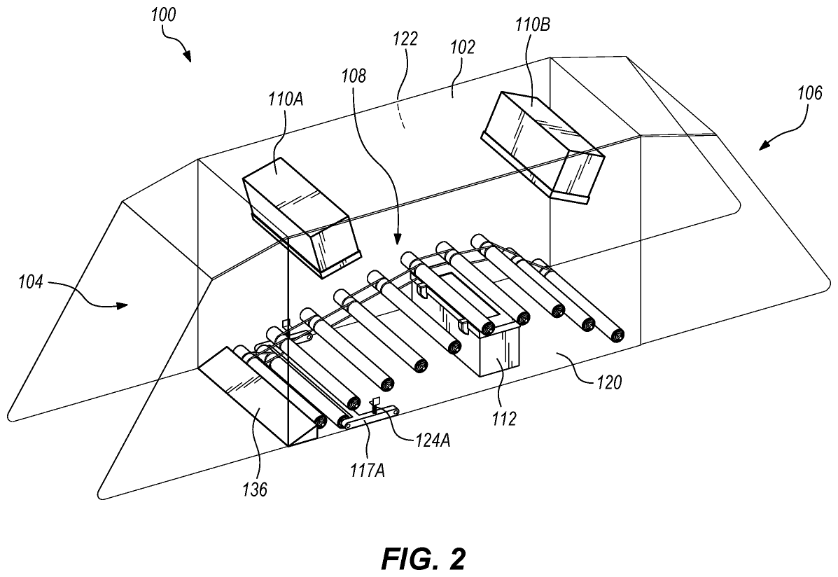

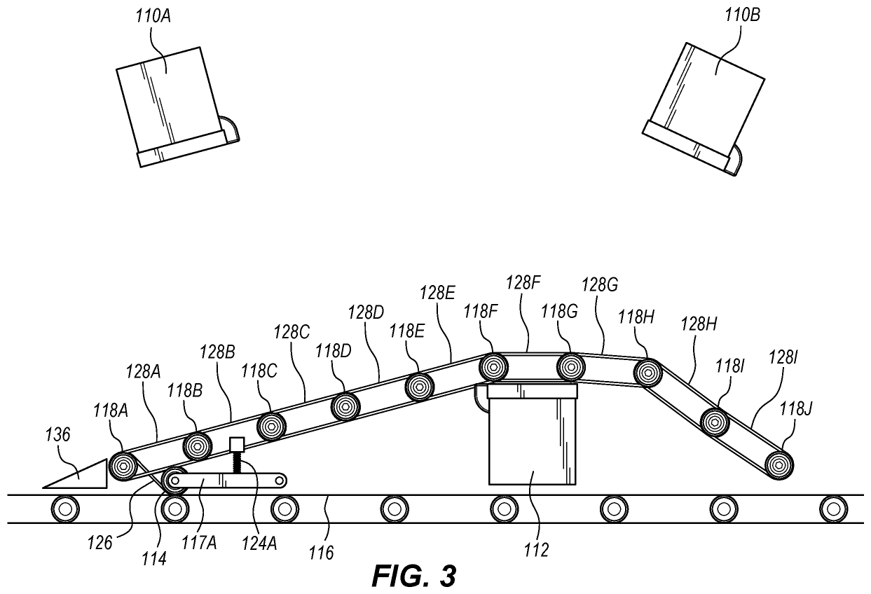

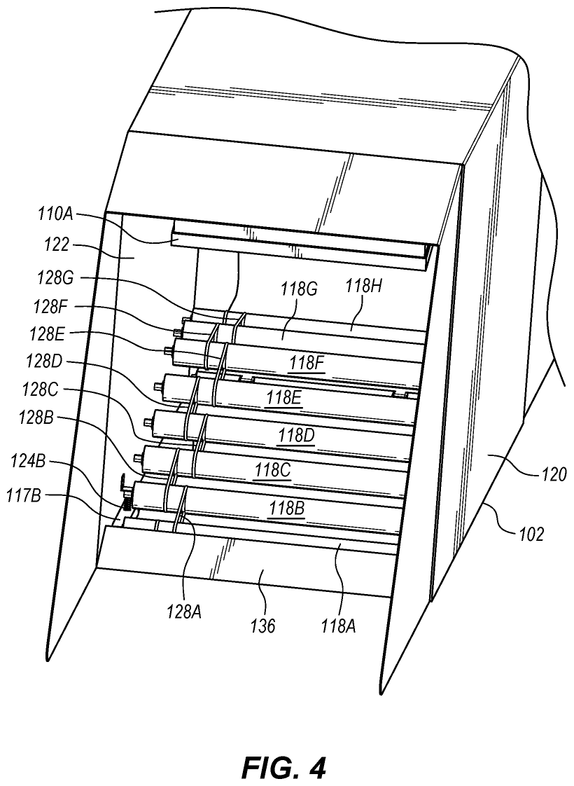

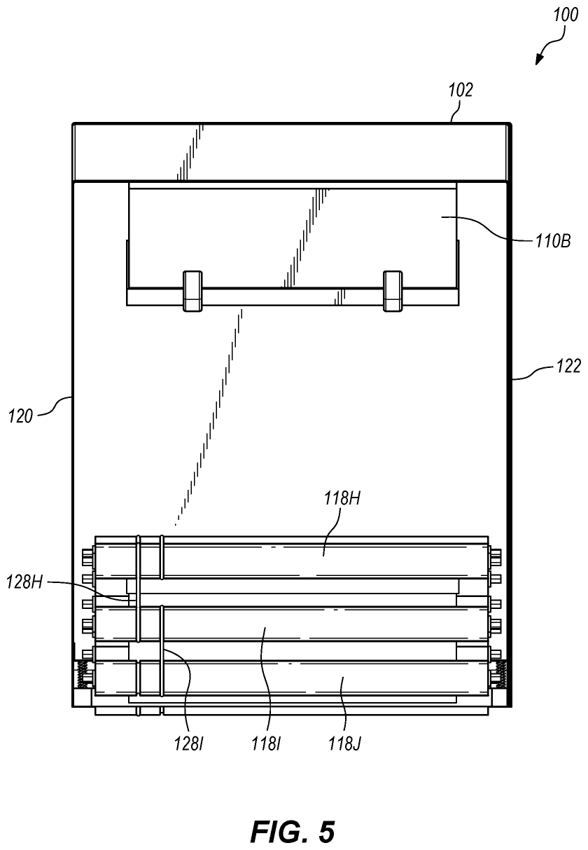

In some embodiments, as shown in FIGS. 1-5, a disinfecting device 100 for conveyors comprises a housing 102, an inlet 104, an outlet 106, a plurality of rollers 108 within the housing 102, and one or more top disinfecting lights 110A-B positioned above the rollers, and at least one bottom disinfecting light 112 positioned beneath the rollers 108. As best seen in FIG. 3, a drive roller 114 is configured to rest directly on, and make contact with, a conveyor belt 116. As the conveyor belt 116 moves in a first direction (from inlet 104 toward outlet 106), it causes the drive roller 114 to rotate in an opposite direction. To ensure the drive roller 114 remains in contact with the conveyor belt 116, it may be spring biased toward the conveyor belt 116. For example, a first support arm 117A may be pivotally coupled to a first sidewall 120 and a second support arm 117B may be pivotally coupled to a second sidewall 122 of the housing 102. The first support arm 117A may be coupled at a first end to the drive roller 114 and pivotally coupled at a second, opposite end to the first sidewall 120, such as by using a bolts, screws, or other fasteners that allow for pivot motion. A first spring 124A may be securely fastened on its top end to the first sidewall 120 to therefore apply a downward force on the first support arm 117A, which forces the drive roller 114 downward to maintain contact with the conveyor belt 116. In like manner, the second support arm 117B may be coupled at a first end to the drive roller 114, on an opposite side of the drive roller 114 from the first support arm 117A, and pivotally coupled at a second, opposite end to the sidewall 122, such as by using bolts, screws, or other fasteners that allow for pivot motion. A second spring 124B applies a downward force on the second support arm 117B, thereby forcing the second support arm 117B to pivot downwardly, forcing the drive roller 114 downward to maintain contact with the conveyor belt 116.

It will be appreciated that the first and second sidewalls 120, 122 rest on an outer surface of the conveyor system and do not make contact with the conveyor belt 116. Accordingly, in some embodiments, only the drive roller 114 makes contact with the conveyor belt 116, as best shown in FIG. 3. The drive roller 114 rotates rearwardly, opposite of the direction of the conveyor belt 116, as a result of the forward motion of the conveyor belt 116 beneath it. Through a series of belts, chains, and/or gears, the drive roller 114 drives the remaining rollers in a forward direction, matching the direction of the conveyor belt 116. For example, drive roller 114 may be coupled to a first roller 118A via a drive belt 126. The drive belt 126 may be in a figure eight configuration so that the first roller 118A rotates in an opposite direction of the drive roller 114 (and in the same direction as the conveyor belt 116). The first roller 118A may then be coupled to each subsequent roller 118B-J through a series of belts 128A-I, thereby ensuring that each roller 118B-J rotates in the direction of the conveyor belt 116. Accordingly, as an item reaches the first roller 118A, it is moved in a forward direction toward roller 118B and to each subsequent roller. It will be appreciated that the rollers 118 are configured at an incline until they pass over bottom disinfection light 112. The rollers 118 may then begin to descend so that the item meets the conveyor belt 116 once more to continue onward.

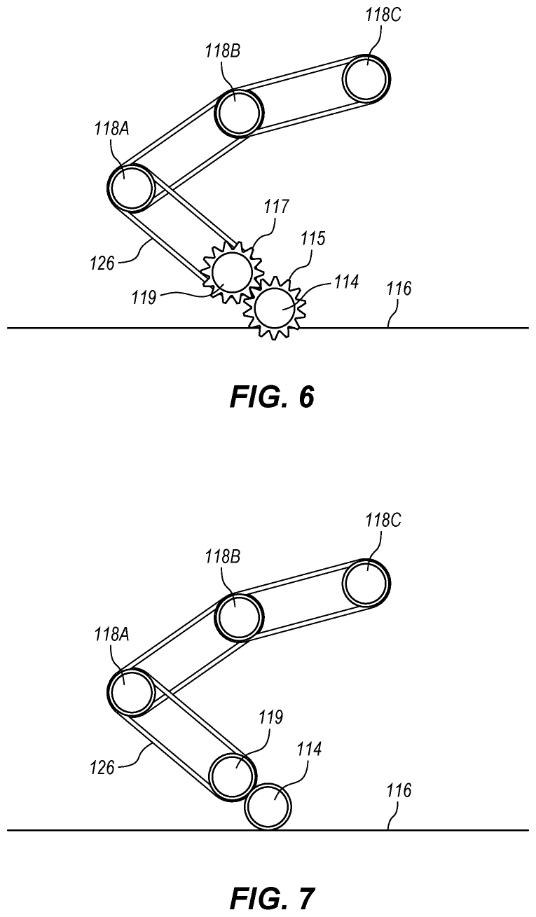

While drive belt 126 is shown as a figure eight belt, it will be appreciated that other configurations may be used to convert the direction of the drive roller 116 (opposite the direction of the conveyor belt 116) so that the subsequent rollers 118B-J rotate in the same direction as the conveyor belt 116. For example, in some embodiments, as shown in FIG. 6, the drive roller 114 may comprise gear teeth 115 for meshing with the gear teeth 117 of an intermediate roller 119, the intermediate roller 119 then coupled to the first roller 118A via a drive belt 126 or chain. In some embodiments, as shown in FIG. 7, the drive roller 114 may be in direct contact with an intermediate roller 119, with the intermediate roller 119 then coupled to the first roller 118A via a drive belt 126 or chain. Rubber surfaces or similar may be used to ensure that the drive roller 114 and intermediate roller 119 rotate in relation to one another. In some embodiments, the drive roller 114 or other rollers 118A-J may couple to the drive system of the conveyor belt 116, such as by coupling the drive roller 114 to a drive roller under the conveyor belt via a belt or chain.

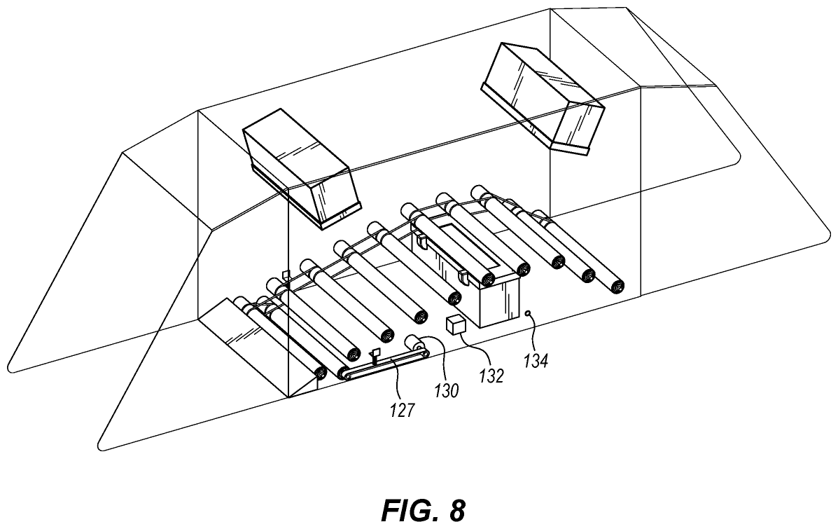

In some embodiments, as shown in FIG. 8, rather than using a drive roller 114 in contact with the conveyor belt 116, the drive roller 114 may be driven by a motor 130. In such a scenario, the drive roller 114 may be driven in the same direction as the conveyor belt 116 via a motor belt 127 or chain so that reversing the direction of subsequent rollers 118A-J is not needed. If motor driven, the speed of the motor 130 may be variable, so that a user may adjust the speed of the plurality of rollers 118 so as to approximate, or match, the speed of the conveyor belt 116. In some embodiments, a microcontroller 132 and a sensor 134 may be used to detect the speed of the conveyor belt 116, with the microcontroller 132 dynamically adjusting the speed of the motor 130 to match the speed of the conveyor belt 116. The sensor 134 disclosed herein may be of any suitable type, including infrared, laser, mechanical switches, or others

In some embodiments, a wedge 136 may be positioned in front of the first roller 118A. The wedge 136 may be triangular in shape, with a first corner adjacent to the conveyor belt 116 and sloping upward toward the first roller 118A, thereby assisting items on the conveyor belt 116 to be lifted onto the first roller 118A where it may then be pulled by the first roller 118A to progress to each subsequent roller 118B-J. The wedge 136 may be in contact with the conveyor belt 116 or close thereto.

In some embodiments, the disinfecting device comprises light-blocking drapes or shields that prevent too much light from being visible to workers who may be working nearby. For example, inlet drapes may aid in preventing light from escaping the housing in a front direction and outlet drapes may aid in preventing light from escaping the housing in a rear direction.

Accordingly, in one method of use, a disinfecting device 100 for conveyors is positioned to rest atop a conveyor belt 116. A drive roller 114 (driven by the conveyor belt 116 or motor 130) drives a plurality of rollers 118A-J. As an item passes down the conveyor belt 116 toward an inlet 104, it is pushed up the wedge 136 and onto the first roller 118A where it is then carried up and over a bottom disinfecting light 112 by the subsequent rollers 118B-J. For example, a plurality of first rollers 118A-F may ascend in sequence to reach a height above the bottom disinfecting light 112. It will be appreciated that the disinfecting light passes between at least rollers 118F and 118G so as to disinfect the underside of the item. A plurality of second rollers 118G-J may descend in sequence so the item falls back to the conveyor belt 116. Top disinfecting lights 110A, 110B disinfect the item from above. Although not shown, additional disinfecting lights may be placed on the interior sides 120, 122 for additional disinfection angles. By placing the disinfecting device 100 atop an existing conveyor belt 116, no modifications to the conveyor belt 116 are required, minimizing down time and while remaining inexpensive to implement. Further, when the drive roller 114 is driven by the conveyor belt 116, the impact to production speed is minimal. It will be appreciated that the disinfecting device 100 may be secured to the conveyor belt 116 via screws, bolts, clamps, or other methods. In some embodiments, if there are not exposed surfaces on the sides of the conveyor belt 116 where the disinfecting device 100 may rest, it will be appreciated that the disinfecting device 100 may comprise a plurality of legs (e.g., one at each corner of the housing 102), which may be height adjustable (e.g., telescopic), so as to be placeable over a conveyor belt 116.

It will be appreciated that the number of rollers 118 may vary, the number of disinfecting lights may vary, and the size and outer formfactor of the housing may vary, without departing herefrom. Further, although generally referred to herein as a “disinfecting device,” it is understood that a disinfecting device of the present disclosure may disinfect, sterilize, sanitize, or otherwise treat and clean the surface of a contaminated item to achieve a lessened state or condition of contamination. Housing 102 may include an exterior material having a first property or function, and an interior material having a second property or function that is different than the first property or function. For example, in at least one embodiment, the housing 102 may include an exterior material that is structurally rigid and opaque, and an interior material that is reflective. In some embodiments, the interior material of the housing 102 may include a coating applied to an inner surface of the exterior material of the housing 102.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A disinfectant device for conveyors, comprising:
a housing having an inlet and an outlet;
a drive roller configured to drive to a plurality of rollers;
a bottom disinfecting light positioned beneath the plurality of rollers, the bottom
light configured to be interposed between the plurality of rollers and a conveyor belt; and
at least one top disinfecting light positioned above the plurality of rollers.

2. The disinfectant device for conveyors of claim 1, wherein the drive roller contacts, and is driven by, the conveyor belt.

3. The disinfectant device for conveyors of claim 2, wherein the drive roller is coupled to a first roller of the plurality of rollers via a drive belt in a figure eight configuration.

4. The disinfectant device for conveyors of claim 1, wherein the drive roller is driven by a motor.

5. The disinfectant device for conveyors of claim 4, further comprising a sensor and a controller configured to control the motor.

6. The disinfectant device for conveyors of claim 5, wherein the sensor and controller are configured to detect a speed of the conveyor belt and to adjust the speed of the motor to match or approximate the speed.

7. The disinfectant device for conveyors of claim 1, wherein the drive roller comprises gear teeth.

8. The disinfectant device for conveyors of claim 7, further comprising an intermediate roller comprising gear teeth configured to mesh with the gear teeth of the drive roller.

9. A disinfectant device for conveyors, comprising:
a housing having an inlet and an outlet, the housing configured to sit atop a conveyor belt;
a drive roller positioned to contact, and be driven by, the conveyor belt;
a plurality of first rollers each ascending in sequence to a position above a bottom disinfecting light;
a plurality of second rollers each descending from a position above the bottom disinfecting light, the bottom disinfecting light positioned between a first roller of the plurality of first rollers and a second roller of the plurality of second rollers, the light configured to shine upwardly therebetween; and
at least one top disinfecting light positioned above the plurality of first and second rollers;
wherein an item is configured to pass over the plurality of first and second rollers wherein the item is interposed between the at least one top disinfecting light and the bottom disinfecting light.

10. The disinfectant device for conveyors of claim 9, wherein the drive roller is coupled to a first roller of the plurality of first rollers via a drive belt in a figure eight configuration.

11. The disinfectant device for conveyors of claim 9, wherein the drive roller comprises gear teeth.

12. The disinfectant device for conveyors of claim 11, further comprising an intermediate roller comprising gear teeth configured to mesh with the gear teeth of the drive roller, the intermediate roller coupled to a first roller of the plurality of first rollers via a belt or chain.

13. A method of disinfecting items on a conveyor belt using a disinfectant device for conveyors, the method comprising:
placing the disinfectant device over a conveyor belt, the conveyor belt interposed between a first sidewall and a second sidewall of a housing, the disinfectant device comprising a wedge at an inlet to carry items from the conveyor belt to a plurality of ascending rollers, the ascending rollers configured to carry the item upward and over a bottom disinfecting light, the bottom disinfecting light interposed between the conveyor belt and the plurality of rollers, the housing further comprising at least one upper disinfecting light, the item interposed between the at least one upper disinfecting light and the bottom disinfecting light; and
wherein after passing over the bottom disinfecting light the item proceeds back to the conveyor belt via a plurality of descending rollers.

14. The method of claim 13, wherein the disinfectant device comprises a drive roller for driving the plurality of ascending rollers and descending rollers.

15. The method of claim 14, wherein the drive roller is in contact with, and driven by, the conveyor belt.

16. The method of claim 15, wherein the drive roller is spring-biased toward the conveyor belt.

17. The method of claim 15, wherein the drive roller comprises gear teeth.

18. The method of claim 17, further comprising an intermediate roller comprising gear teeth configured to mesh with the geared teeth of the drive roller.

19. The method of claim 13, wherein the drive roller is motor driven.
